(19) United States
(12) Patent Application Publication
(10) Pub. No.: US 2008/0208342 A1
(43) Pub. Date: Aug. 28, 2008

(54) SPINAL IMPLANT

(75) Inventors: David A. Hanson, Urbandale, IA (US); Christopher J. Valois, Champlin, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/763,619

(22) Filed: Jun. 15, 2007

Related U.S. Application Data
(62) Division of application No. 29/277,464, filed on Feb. 27, 2007, now abandoned.
Application No. 29/277,464 is a division of application No. 29/277,466, filed on Feb. 27, 2007, now abandoned.

(51) Int. Cl. A61F 2/44

(52) U.S. Cl. 623/17.16

(57) ABSTRACT

An implant is provided that includes a body having a convex and tapered leading end, a convex trailing end, opposing generally planar sides extending between the leading and trailing ends and superior and inferior surfaces. An opening extends through the body between the superior and inferior surfaces. The implant further includes a plurality of protrusions provided on each of the superior and inferior surfaces. Each protrusion has a flattened distal surface. The leading end includes first and second inclined surfaces and an intermediate surface, with the first inclined surface sloping downward from the superior surface to the intermediate surface and the second inclined surface sloping upward from the inferior surface to the intermediate surface.

### CROSS REFERENCE

This application claims priority to U.S. Design patent application Ser. No. 29/277,464, “Spinal Implant”, filed Feb. 27, 2007; and U.S. Design patent application Ser. No. 29/277,466, “Spinal Implant”, filed Feb. 27, 2007, each disclosure of which is expressly incorporated by reference herein in its entirety.

### FIELD OF THE INVENTION

The present invention relates generally to skeletal implants. More particularly, the present invention relates to implants for stabilizing intervertebral joints.

### BACKGROUND OF THE INVENTION

Chronic back problems cause pain and disability for a large segment of the population. In many cases, chronic back problems are caused by intervertebral disc disease. When an intervertebral disc is diseased, the vertebrae between which the disc is positioned may be inadequately supported, resulting in persistent pain. Stabilization and/or arthrodesis of the intervertebral joint can reduce the pain and debilitating effects associated with disc disease.

Spinal stabilization systems and procedures have been developed to stabilize diseased intervertebral joints and, in some cases, to fuse the vertebrae that are adjacent the diseased joint space. Most fusion techniques include removing some or all of the disc material from the affected joint, and stabilizing the joint by inserting an implant (e.g., a bone graft or other material to facilitate fusion of the vertebrae) in the cleaned intervertebral space.

Spinal implants can be inserted into the intervertebral space through an anterior approach, a lateral (transverse) approach, a posterior approach, or postero-lateral approach. The anterior approach involves a surgeon seeking access to the spine through the front (i.e., abdominal area) of the patient. The posterior approach involves a surgeon seeking access to the spine through the back of the patient. The postero-lateral approach is similar to the posterior approach with access coming more from either or both sides of the patient. A variety of different anterior, posterior and postero-lateral techniques are known.

### SUMMARY OF THE INVENTION

While the invention will be described in connection with certain embodiments, it will be understood that the invention is not limited to these embodiments. On the contrary, the invention includes all alternatives, modifications and equivalents as may be included within the spirit and scope of the present invention.

An implant is provided comprising a body having a convex and tapered leading end, a convex trailing end and opposing generally planar sides extending between the leading and trailing ends. The body also includes superior and inferior surfaces and an opening that extends through the body between the superior and inferior surfaces. The implant further includes a plurality of protrusions provided on each of the superior and inferior surfaces. Each protrusion has a flattened distal surface. The leading end includes first and second inclined surfaces and an intermediate surface, with the first inclined surface sloping downwardly from the superior surface to the intermediate surface and the second inclined surface sloping upwardly from the inferior surface to the intermediate surface.

In one embodiment, the superior and inferior surfaces are parallel to one another. In another embodiment, the superior and inferior surfaces diverge away from one another between the leading and trailing ends.

### BRIEF DESCRIPTION OF THE DRAWINGS

The accompanying drawings, which are incorporated in and constitute a part of this specification, illustrate embodiments of the invention and, together with a general description of the invention given above, and a detailed description of the embodiments given below, serve to explain the principles of the invention.

FIG. 1 is a perspective view illustrating a spinal implant according to one embodiment of the present invention;

FIG. 2 is a side elevation view of the implant shown in FIG. 1;

FIG. 3 is an enlarged view of the encircled portion of FIG. 2;

FIG. 4 is a top view of the implant shown in FIGS. 1-3;

FIG. 5 is an elevation view of the leading end of the implant shown in FIGS. 1-4;

FIG. 6 is an elevation view of the trailing end of the implant shown in FIGS. 1-5;

FIG. 7 is a perspective view of a spinal implant according to another embodiment of the present invention;

FIG. 8 is a side elevation view of the implant shown in FIG. 7;

FIG. 9 is a top view of the implant shown in FIGS. 7-8;

FIG. 10 is an elevation view of the leading end of the implant shown in FIGS. 7-9; and

FIG. 11 is an elevation view of the trailing end of the implant shown in FIGS. 7-10.

### DETAILED DESCRIPTION OF THE INVENTION

The present invention is directed to skeletal implants and methods for placing implants between bones and fusing the bones together. It is preferred for the implants to be used for vertebral/spinal applications such as fusing cervical, thoracic and/or lumbar intervertebral joints. In the case of fusing an intervertebral joint, implants in accordance with the principles of the present invention can be implanted using an anterior, posterior or postero-lateral approach to the patient's vertebrae.

As used herein, an “implant” includes any implant suitable for facilitating fusion between adjacent bones and includes implants prepared from known implant materials including, non-bone material such as titanium, stainless steel, porous tantalum or other metal, bio-glass, calcium phosphate, ceramic, carbon fiber-based polymers and biodegradable polymers. However, it is preferred for implants in accordance with the principles of the present invention to be derived from natural bone tissue (e.g., allograft and xenograft bone). It is most preferred for implants in accordance with the principles of the present invention to be derived from natural bone such as from a cadaveric allograft bone source. For example, the implants can be derived by cross-sectioning cortical rings from cadaveric allograft long bones such as femur, tibia or fibula or from other bone sources such as the illium. Alternatively, the implants can be formed/molded from ground, sintered or composite bone material. Xenograft bones (e.g., from a bovine source) also can be used.

The term “allograft” will be understood to mean a bone implant from a donor transplanted to a genetically dissimilar recipient of the same species. The term “xenograft” will be understood to mean a bone implant from a donor transplanted to a recipient of a different species.

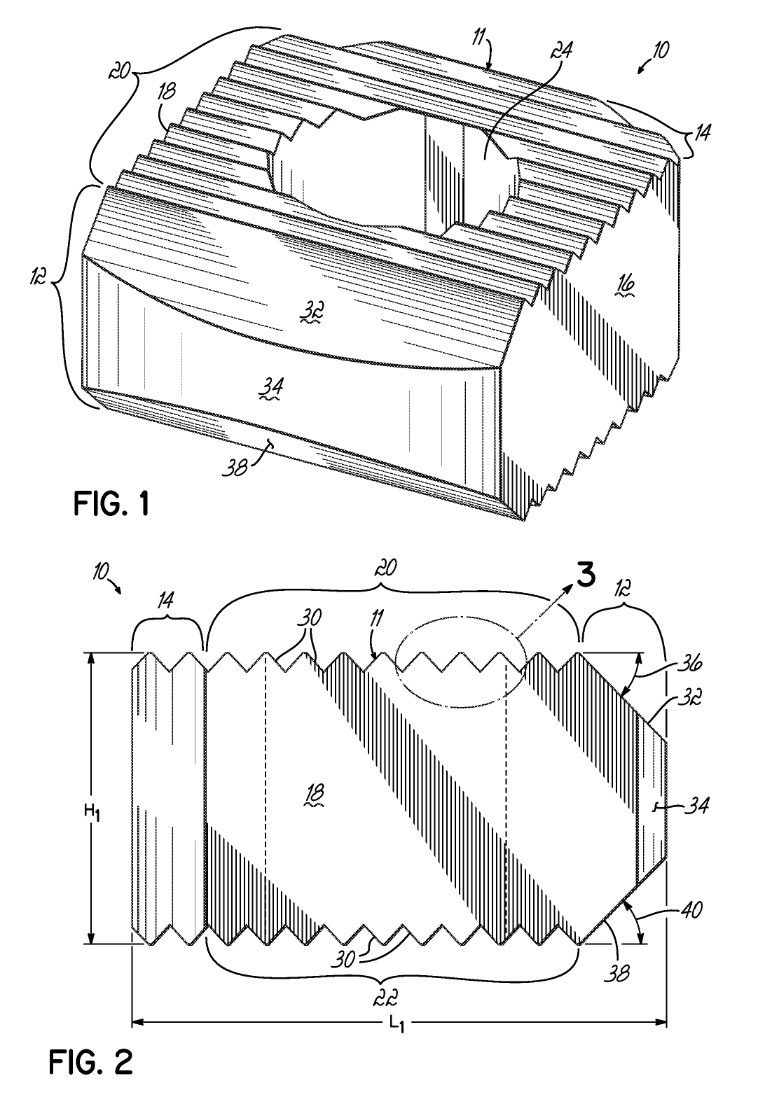

FIGS. 1-6 illustrate a spinal implant 10 that may be made as a one-piece, or unitary, construction. In one embodiment, implant 10 is made from allograft bone. However, implant 10 may be made from other suitable biocompatible materials including those discussed previously. Implant 10 may be derived from a bone source such as the anterior tibia. Alternatively, the implant 10 can be derived from the transverse cross-section of cadaver bone, such as from the femur, and includes a cortical ring. After the relatively soft tissue and marrow is removed from the interior of the ring using conventional procedures, the cortical ring is shaped using conventional procedures to provide the desired configuration of implant 10.

In one embodiment, implant 10 is used to fuse adjacent cervical vertebrae and is inserted between the adjacent vertebrae. However, in other embodiments implant 10 may be used to fuse thoracic and/or lumbar intervertebral joints. Implant 10 includes a body 11 having a convex and tapered leading end 12, a convex trailing end 14 and opposing planar sides 16 and 18 extending between the leading 12 and trailing 14 ends. Although implants according to the principles of the present invention may be inserted between adjacent vertebrae using various approaches, implant 10 is inserted using an anterior approach in the exemplary embodiment. Accordingly, leading end 12 is the posterior end and trailing end 14 is the anterior end when inserted between adjacent vertebrae. Body 11 further includes a superior surface 20 that extends between leading 12 and trailing 14 ends and between sides 16, 18. Body 11 also includes an inferior surface 22 that is parallel to superior surface 20. Inferior surface 22 also extends between leading 12 and trailing 14 ends and between sides 16, 18.

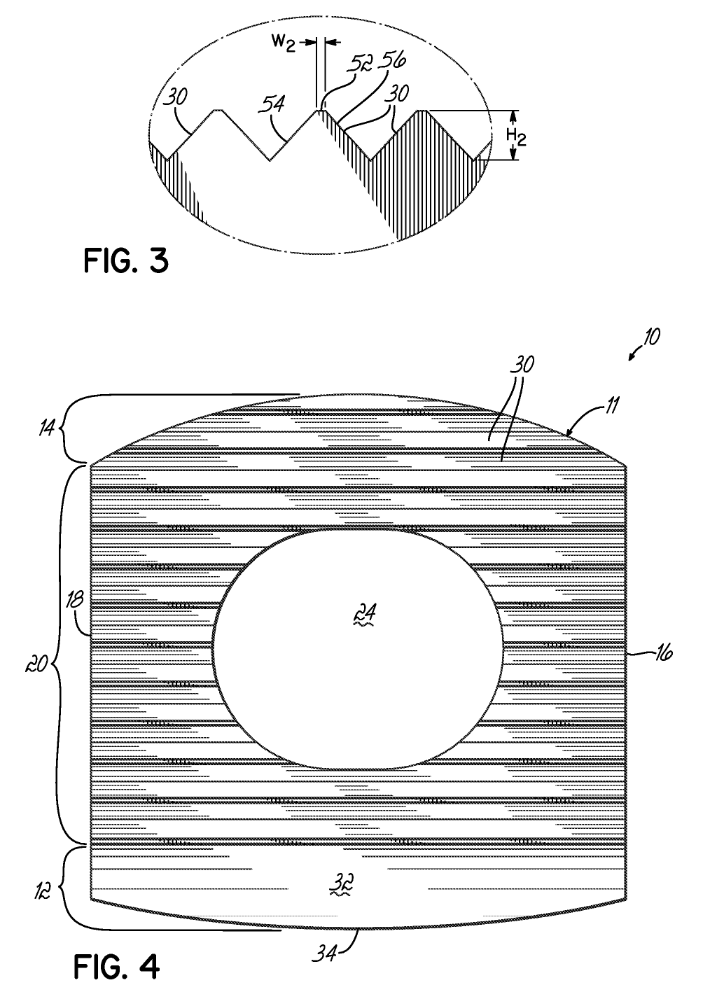

Implant 10 includes an opening 24 that extends through body 11 between superior surface 20 and inferior surface 22 and also includes a plurality of protrusions 30 provided on each of the superior surface 20 and the inferior surface 22 as will be described in greater detail below in connection with FIG. 3.

Leading end 12 may be chamfered on top and bottom to facilitate the insertion of implant 10 between adjacent vertebrae. In the illustrated embodiment, leading end 12 includes an inclined surface 32 that slopes downwardly away from superior surface 20 to an intermediate surface 34, which is a posterior surface, and is inclined at an angle 36 relative to superior surface 20. Leading end 12 further includes an inclined surface 38 that slopes upwardly away from inferior surface 22 to intermediate surface 34 and is inclined at an angle 40 relative to inferior surface 22. The magnitude of angles 36 and 40 may vary from 0 degrees to about 60 degrees. More preferably, the magnitude of angles 36 and 40 may vary from about 20 degrees to about 45 degrees.

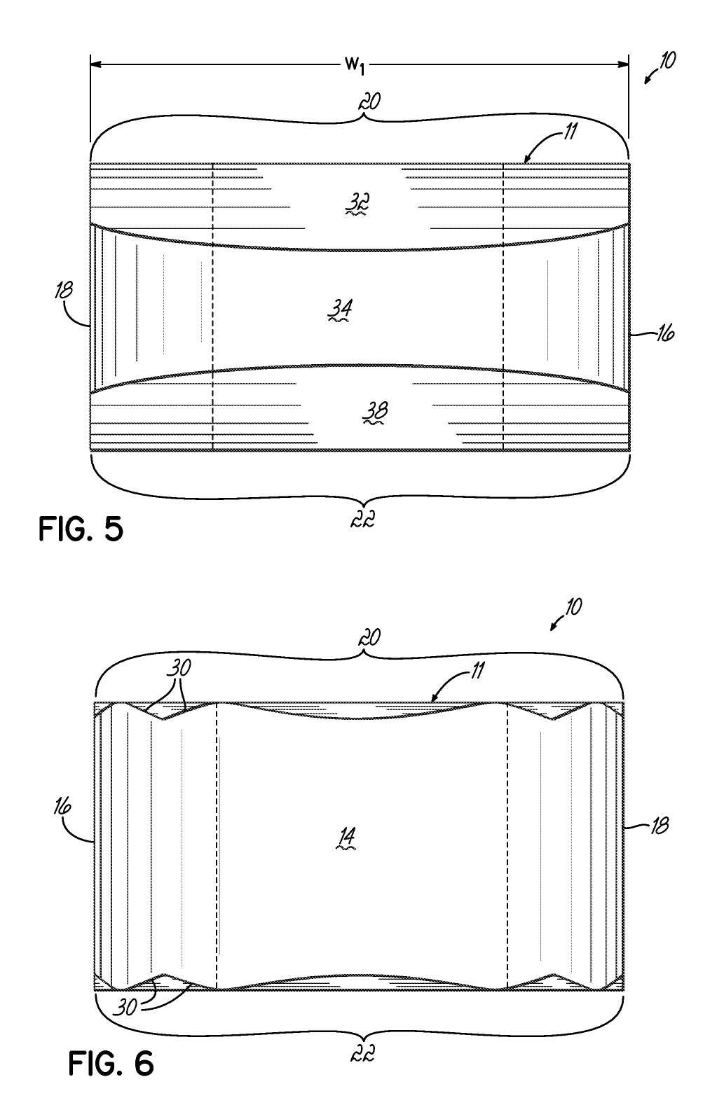

Implant 10 has a height “H₁” (FIG. 2), a length “L₁” (FIG. 2) and a width “W₁” (FIG. 5). Height “H₁” extends between the superior surface 20 and the inferior surface 22 and may have a magnitude ranging from about 5 mm to about 10 mm. Length “L₁” extends between the leading end 12 and trailing end 14 and may have a magnitude ranging from about 10 mm to about 15 mm. Width “W₁” extends between sides 16, 18 and may have a magnitude ranging from about 10 mm to about 15 mm. More preferably, the magnitudes of “L₁” and “W₁” may range from about 11 mm to about 14 mm.

Referring to FIG. 3, each protrusion 30 has a flattened distal surface 52 and side surfaces 54, 56 that converge toward one another from the respective one of superior surface 20 and inferior surface 22 to flattened surface 52. As used in the above description, distal refers to the surface of protrusion 30 that is the farthest from the respective one of superior surface 20 and inferior surface 22. The shape of each protrusion 30 has a surface area that enhances bony ingrowth. Flattened distal surface 52 has a width “W₂” that may range in magnitude from about 0.04 mm to about 0.12 mm. Protrusion 30 has a height “H₂” that may range in magnitude from about 0.26 mm to about 0.46 mm.

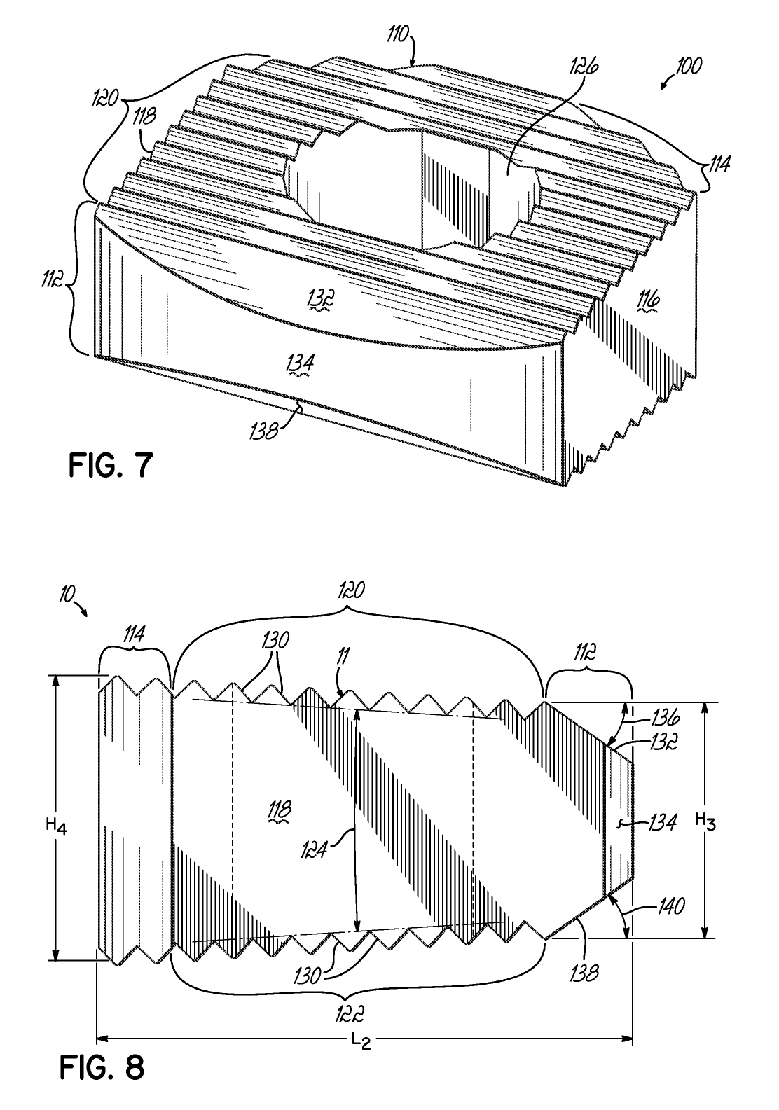

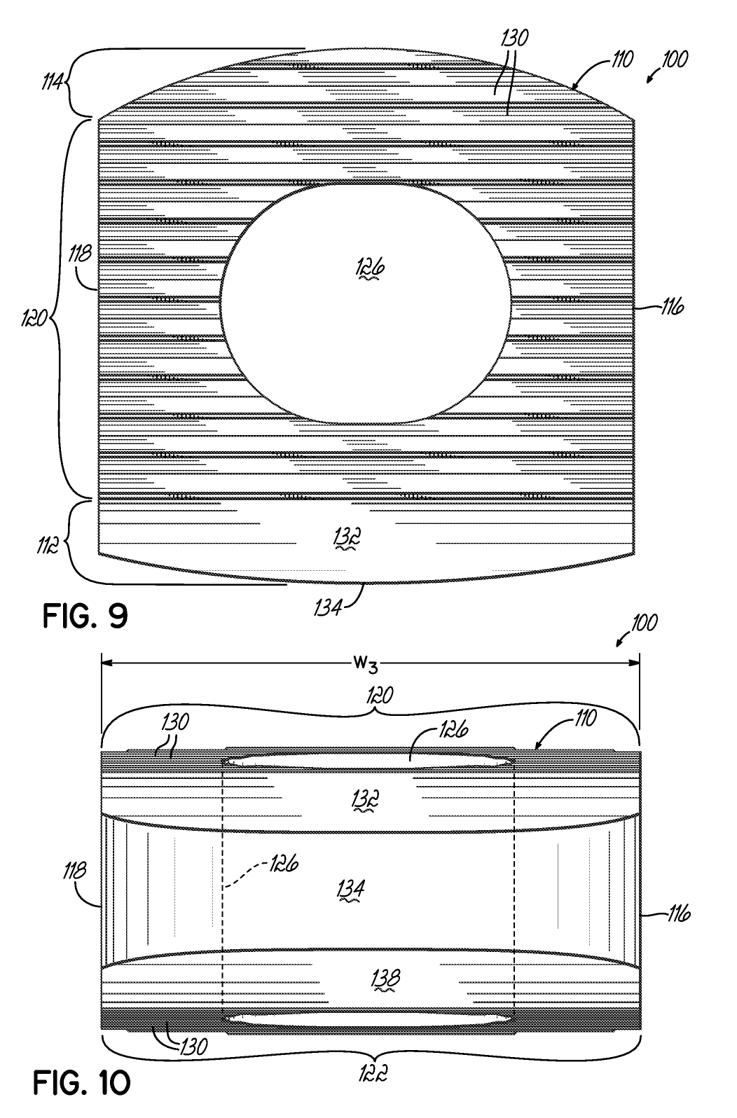

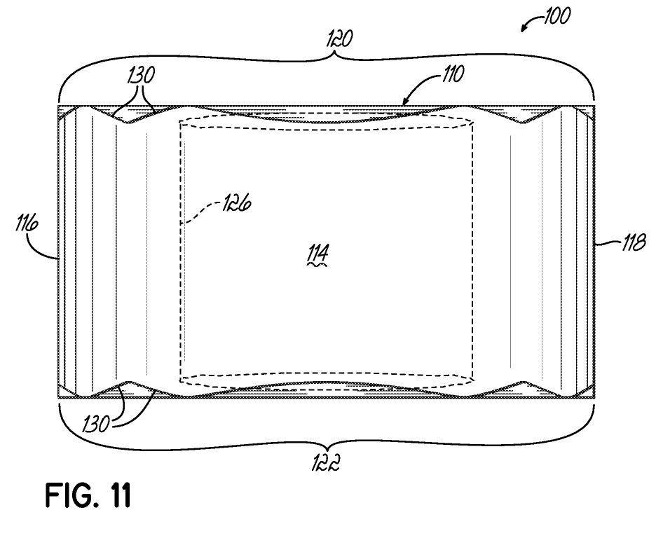

FIGS. 7-11 illustrate a spinal implant 100 according to another embodiment of the present invention. Implant 100 is similar to implant 10 except that the subsequently discussed superior and inferior surfaces of implant 100 are inclined relative to one another, instead of parallel to one another. Similar to implant 10, implant 100 may be made as a one-piece, or unitary, construction from allograft bone, or other suitable biocompatible materials including those discussed previously. In one embodiment, implant 100 is used to fuse adjacent cervical vertebrae and is inserted between the adjacent vertebrae. However, in other embodiments implant 100 may be used to fuse thoracic and/or lumbar joints.

Implant 100 includes a body 110 having a convex and tapered leading end 112, a convex trailing end 114 and opposed planar sides 116 and 118 extending between the leading 112 and trailing 114 ends. In the exemplary embodiment, implant 100 is inserted using an anterior approach. Accordingly, leading end 112 is the posterior end and trailing end 114 is the anterior end when inserted between adjacent vertebrae.

Body 110 further includes a superior surface 120 that extends between leading 112 and trailing 114 ends and between sides 116, 118. Superior surface 120 is inclined upwardly from the leading end 112 to the trailing end 114. Body 110 also includes an inferior surface 122 that extends between leading 112 and trailing 114 ends and between sides 116, 118. Inferior surface 122 is inclined downwardly from leading end 112 to trailing end 114. Accordingly, body 110 has an included angle 124, which is a lordotic angle, between the superior 120 and inferior 122 surfaces. Angle 124 may have a magnitude ranging from about 6 degrees to about 8 degrees to accommodate lordosis of the spine. Also, as subsequently discussed in conjunction with FIG. 8, the height of implant 100 at the trailing, or anterior, end 114 is greater than the height of implant 100 at the leading, or posterior, end 112.

Implant 100 includes an opening 126 that extends between superior surface 120 and inferior surface 122. A plurality of the protrusions 130 are provided on each of the superior surface 120 and the inferior surface 122. In one embodiment, protrusions 130 may have the same shape and size as protrusions 30 of implant 10 described above.

Leading end 112 may be chamfered on top and bottom to facilitate the insertion of implant 100 between adjacent vertebrae. Leading end 112 includes an inclined surface 132 that slopes downwardly away from superior surface 120 to an intermediate surface 134, which is a posterior surface, and is inclined at an angle 136 relative to superior surface 120. Leading end 112 further includes an inclined surface 138 that slopes upwardly away from inferior surface 122 to intermediate surface 134 and is inclined at an angle 140 relative to inferior surface 122. The magnitude of angles 136 and 140 may be the same as those discussed previously for angles 36, 40, respectively.

Implant 100 has a height “H₃” that extends between the superior surface 120 and the inferior surface 122 at the leading end 112. Implant 100 further includes a height “H₄” that extends between the superior surface 120 and the inferior surface 122 at the trailing end 114. As shown in FIG. 8, height “H₄” is greater than height “H₃” to accommodate lordosis of the spine. Implant 110 also includes a length “L₂” (FIG. 8) and a width “W₃” (FIG. 10). Length “L₂” extends between the leading end 112 and trailing end 114 and may have a magnitude ranging from about 10 mm to about 15 mm. Width “W₃” extends between sides 116, 118 and may have a magnitude ranging from about 10 mm to about 15 mm. More preferably, “L₂” and “W₃” may have magnitudes ranging from about 11 mm to about 14 mm. Height “H₃” may have a magnitude ranging from about 5 mm to about 10 mm. The magnitude of height “H₄” may be determined from the particular values of height “H₃”, the lordotic angle 124 and length “L₂”.

The implants 10, 100 can be inserted by a variety of surgical approaches, including, but not limited to an anterior approach, a lateral (transverse) approach, a posterior approach, or postero-lateral approach by engaging the implants 10, 100 with an instrument, such as an inserter. The implants 10, 100 can include grooves, indentations, slots or other surface deficits that allow the inserter to engage the implants 10, 100. For example, the trailing end 14, 114 of the implant can include holes, such as a circular hole or holes that mate with prongs on the inserter. Alternatively, the trailing end 14, 114 can include two or more square or rectangular surface deficits cut into the superior 20, 120 and inferior surfaces 22, 122 that can be engaged by the inserter. In other embodiments, slots or grooves can be formed in the each of the sides 16, 18 and 116, 118. The slots or grooves can be partially formed into and engaged at the trailing end 14, 114 by the inserter. The slots or grooves can be formed such that a portion of the implant 10, 110 forms a positive stop for the inserter instrument. Alternatively, the slots or grooves can extend the entire length of the sides 16, 18 and 116, 118.

While the present invention has been illustrated by the description of and exemplary embodiments thereof, and while the embodiments have been described in considerable detail, it is not intended to restrict or in any way limit the scope of the appended claims to such detail. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. An implant comprising:
a body having a convex and tapered leading end, a convex trailing end and opposing generally planar sides extending between said leading and trailing ends, said body further including superior and inferior surfaces;
an opening extending through said body between said superior and inferior surfaces;
a plurality of protrusions provided on each of said superior and inferior surfaces, each said protrusion having a flattened distal surface;
said leading end includes first and second inclined surfaces and an intermediate surface, said first inclined surface sloping downwardly from said superior surface to said intermediate surface, said second inclined surface sloping upwardly from said inferior surface to said intermediate surface.

2. The implant of claim 1, wherein:
said body has a width ranging from about 11 mm to about 14 mm, a length ranging from about 11 mm to about 14 mm, and a height ranging from about 5 mm to about 10 mm.

3. The implant of claim 2, wherein:
said flattened distal surface has a width ranging from about 0.04 mm to about 0.12 mm, each of said protrusions having a height ranging from about 0.26 mm to about 0.46 mm.

4. The implant of claim 1, wherein:
said superior and inferior surfaces are parallel to one another.

5. The implant of claim 1, wherein:
said superior and inferior surfaces diverge away from one another between said leading end and said trailing end, said implant having an included angle between said superior and inferior surfaces ranging from about 6 degrees to about 8 degrees.
